(19) United States
(12) Patent Application Publication
(10) Pub. No.: US 2002/0143373 A1
(43) Pub. Date: Oct. 3, 2002

(54) System and method for therapeutic application of energy

(76) Inventors: Peter A. Courtnage, Anchorage, AK (US); Robin E. Schaffer, Englewood, CO (US)

Correspondence Address: LATHROP & GAGE LC, 4845 PEARL EAST CIRCLE, SUITE 300, BOULDER, CO, 80301, US

(21) Appl. No.: 10/057,512

(22) Filed: Jan. 25, 2002

Related U.S. Application Data
(60) Provisional application No. 60/264,115, filed on Jan. 25, 2001.

(51) Int. Cl. A61N 5/00; A61N 5/067

(52) U.S. Cl. 607/91; 607/89; 607/100; 607/115; 607/148; 607/152

(57) ABSTRACT

A therapeutic device system provides therapeutic application of energy to a living body. The energy is applied by the action of photon-emitting diodes, and photon emitting diodes in combination with transcutaneous electrical stimulators. A power grid is adapted to provide electrical current for operation of the energy sources. A shapable housing contains the energy sources and the power grid. The shapable housing is selectively moldable with memory retention to retain a shape configuration, and may be cast or adapted conformably as a custom or generically sized device over a treatment area and placed on the living body. Program instructions provide for implementation of a plurality of therapeutic modalities.

### RELATED APPLICATIONS

This application claims benefit of priority to provisional application serial number 60/264,115 filed Jan. 25, 2001, which is hereby incorporated by reference to the same extent as though fully replicated herein.

### BACKGROUND OF THE INVENTION

1. Field of the Invention

The invention pertains to the field of photodynamic therapy and, more particularly, to systems that emit light to enhance natural healing processes in situations where light is beneficial to such processes.

2. Description of the Related Art

Utilization of light, heat, and electro-stimulation has a long history in both oriental and western medicine. The earliest western historical record of the use of light, dates back to Herodotus, in the 6^{th} century B.C.E., with his observation that normal bone growth requires exposure to sunlight. Later, Greek and Roman physicians employed light in treating a variety of conditions, such as epilepsy, arthritis, asthma and obesity, and as preventive medicine. Interest in light therapies rekindled in the 18^{th} and 19^{th} centuries, in part, due to the increase in illnesses caused by lack of light and crowded conditions in urban settings. Sunbaths were recommended for rickets, edema, scurvy, dropsy, rheumatic arthritis and depression.

In 1923, Alexander Gurwitsch, a Russian biologist observed that living cells emit “mitogenetic radiation,” which is non-thermal electromagnetic radiation that is associated with important biological processes. Healthy cells are described as emitting a wavelength of 625-to-700 nanometers, whereas pathological or ill cells emit shorter wavelengths. In 1966, Endre Mester, a Hungarian, performed a series of in vitro and in vivo studies which verified the positive effects of low intensity laser light. He demonstrated that laser formed light, at low intensity, accelerated tissue healing, increased collagen synthesis, promoted the formation of new blood vessels, and enhanced enzyme synthesis. The first laser therapy clinic was established in Budapest in 1967. One example of a laser therapeutic apparatus is U.S. Pat. No. 5,150,704 to Tatebayashi et al., which shows laser probes mounted on a support table having a selective position lock mechanism for the laser probes.

There have now been over 3,000 studies performed evaluating the effectiveness, efficacy, and applicability of low level lasers. More than 100 double-blind studies support the use of low level therapeutic laser applications for the treatment of a wide variety of conditions. Research has demonstrated that low level therapeutic lasers stimulate mitochondrial activity, enhance ATP production, increase production of singlet oxygen, stimulate DNA and RNA synthesis, stimulate repair and regeneration of central and peripheral nerve damage, increase protein synthesis, accelerate cellular metabolism, enhance the repair of acute and chronic ulcerations and wounds, increase circulation, enhance revascularization, reduce inflammation, and speed recovery from repetitive motion injuries, such as carpel tunnel syndrome.

The coherent light of lasers is not the only means by which light can influence the healing of the human body. Studies of human cells under conditions of microgravity and hypergravity reveal that there is a direct linear correlation between the increase and decrease of the gravitational field and resulting cellular function. NASA-funded research has shown that light-emitting diodes (LEDs) enhance cellular function, even under conditions of microgravity and hypergravity. This research demonstrated that LEDs, like lasers, catalyze increased mitochondrial activity, thereby enhancing the physiological function of cells and, collectively, tissues formed from these cells. This research further demonstrated that LEDs, like lasers, enhance DNA synthesis in fibroblasts. Muscle cells were shown to quintuple their growth in a single application combining 680, 730, and 880 nanometer LEDs operating at an exposure of four joules per centimeter squared. Examples of light therapy systems using the incoherent light of LEDs include U.S. Pat. Nos. 4,930,504 to Diamantopoulos et al., 5,259,380 to Mendes at al., 5,800,479 to Thiberg, 6,063,108 to Salansky et al., and 6,107,466 to Hasan et al. The use of laser diodes is associated with a faster tissue response curve than occurs with LEDs, but laser diodes are far more expensive than LEDs.

Another technique for stimulating natural healing processes is the use of trans-cutaneous electro-stimulation, for example, as described in U.S. Pat. No. 4,676,246 to Korenaga. Finnish research has demonstrated that therapeutic protocols using trans-cutaneous electro-stimulation combined with laser application enhances the therapeutic outcome. This therapeutic enhancement results from increased neural, vascular and lymphatic circulation, which is improved when compared to the therapeutic benefit of light application alone. The effects of LEDs in combination with trans-cutaneous electro stimulation have yet to be evaluated in formal research.

While the therapeutic benefits of light, heat, and electro-stimulation are generally known, there is a growing body of evidence to suggest that different treatment regimens are appropriate for different types of conditions that include, for example, surgical scars, trauma scars, infections, the promotion of lactation, sprains, tears, and chronic repetitive stress syndrome. Available devices are unable to deliver treatments for this array of conditions.

### SUMMARY OF THE INVENTION

The present invention overcomes the problems that are outlined above and advances the art by providing a single system that combines physical and logical elements that are capable of treating a wide variety of conditions to achieve the therapeutic benefits of light, heat, and/or electro-stimulation.

The system generally comprises energy sources, such as photon-emitting diodes (LEDs), laser diodes, and trans-cutaneous electrical stimulators, which may be used in any combination. A power grid is adapted to provide electrical current for operation of the energy sources. A shapable housing is provided for the energy sources, and permits coupling of the energy sources with the power grid. The shapable housing may be selectively moldable to retain a shape configuration when adapted conformably over a treatment area and placed on the living body.

In various embodiments, the shapable housing may comprise a flexible material that conforms to the treatment area. The flexible material may include a metal sheet or mesh, such as a copper mesh or a lead sheet. Alternatively, a curable material may be used, such as a thermosetting resin or casting material, that is flexible until cured and, after curing, comprises a substantially rigid material that provides support to the living body. For example, the thermosetting resin may become flexible when heated to a temperature above a design temperature and cure or harden to rigidity when cooled to a temperature below the design temperature.

In the case of a casting material, the shapable housing may be applied as a resin-mesh composite that is initially in a flexible state which converts to a substantially rigid state upon curing of the resin. Accordingly, the shapable housing may be adjusted to implement a casting modality for treatment of an injury to the living body.

Metal meshes, sheet deformable metal sheets, or putty polymers, impart the shapable housing with shape-memory retention, such that the material is capable of being reshaped to a neutral state after treatment on the living body is concluded. Other materials that are useful in this regard include manually deformable materials, heat setting materials, and chemical setting materials.

In accord with various instrumentalities of the system, the shapable housing is adaptable to a portion of the living body. An adhesive layer may be applied to the shapable housing to promote contact with the living body. The shapable housing may be adapted by molding, on a customized basis, to mirror individualized templates taken from surgical wounds; surgical scars; trauma-induced scars; infection scarring; skin lesions; abscesses; ulcerations; tumors; cysts; and physiological abnormalities related to soft-tissue, organ, lymph, neurological or vascular compromise of the living body. Similarly, the shapable housing may be adapted on a custom basis to mirror individualized templates taken of anatomical zones of the living body, such as breasts, joints, limbs, neck, and the torso. Alternatively the shapable housing may be generically-sized for the aforementioned purposes. The shapable housing may also be configured for entry into an enclosure of the living body, such as an open wound, an ear opening, a nasal cavity, throat cavity, or even an anal cavity for the promotion of natural healing or infection-fighting processes. In such situations, the shapable housing may be adapted to provide three-dimensional exposure to the energy sources.

The energy sources, for example, may comprise both photon-emitting sources and trans-cutaneous electrical stimulators. The photon-emitting sources may be embedded in the shapable housing, which can be formed of an insulating material to enhance heat concentration over the treatment area. Alternatively, the shapable housing may be formed of a heat conductive material to dissipate heat over the treatment area. The photon-emitting sources may be covered by an optically transparent protective layer that provides a surface interfacing with the skin. This surface can be cleaned and disinfected or sterilized, which eliminates a problem in prior art devices having diodes that are exposed to the skin or wound site.

A power source capable of activating the energy sources is connected to the power grid. The power source may be self-contained in the shapable housing. For example, a battery may function as the power source, and the battery may be a chemical battery having a flexible structural composition which is conformable to the treatment area. A voltage regulator is useful for uniform distribution of electrical current to the energy sources. The power grid may be embedded in the shapable housing.

A control mechanism, such as a central processor (CPU) configured with program instructions, may be utilized for regulation of output from the energy sources. User-selectable functions may be provided to the control mechanism, for example, by the provision of manual switches for triggering operation of program instructions that are processed by the CPU. The program instructions may, for example, reside on programmable memory that is operably coupled with the CPU. The program instructions may include therapeutic control instructions for implementing a variety of therapeutic modalites for use in treating various conditions through use of the energy sources. A telecommunications linkage may also be used for remotely selecting a therapeutic modality and retrieving a record of the therapeutic modalities that have been implemented on the living body.

As mentioned above, the various therapeutic modalities under the control of the program instructions may, for example, include combined treatment modalities that sequence different pattern activation of the energy sources within a single therapeutic application. This type of combined modality treatment may be useful, for example, in treating a wound site that has become infected, or a muscle tear that is also associated with ligament damage. Similarly, a plurality of shapable housings may be placed under the control of a single processor, which may be configured to implement different treatment modalities for the respective shapable housings.

Other treatment modalities that may be selectively accessed from the control mechanism include selective definition of total elapse exposure time of the energy sources, selection of a wave form for the electrical current used in energizing the energy sources (e.g., sine wave, square wave, or sawtooth wave), voltage selection, and setting the frequency modulation (Hz) of the energy sources within the shapable housing. Where the energy sources comprise trans-cutaneous electro stimulators, the control mechanism may permit a user to select whether the trans-cutaneous electro stimulators use micro-electrical current or macro-electrical current to energize the trans-cutaneous electro-stimulators. Furthermore, a user may select the type of energy sources for activation within the shapable housing, e.g., as between LEDs, laser diodes, or trans-cutaneous electro-stimulators. Among the respective light sources, the user may further select desired wavelengths for emission upon activation of a corresponding portion of the light sources. The control mechanism may further permit user selection for milliwatts of the electrical current that is applied to the respective energy sources, or Joules of photon emission from the energy sources.

The control mechanism may include a visual display that is configured to provide visual confirmation of the selected program instructions and the therapeutic modality that is being implemented. A speech generator, e.g., a speech synthesizer or recording playback mechanism may be used to generate interval auditory reminders of the system activity status according to the program instructions.

The shapable housing may also be provided with other functionalities, such as those that record and measure the effects of treatment. In the various system instrumentalities, the system may permit electromyographic reporting of the electrical skin conductivity of the treatment area. Thermographic reporting of skin temperature alterations over the treatment area may also be obtained. These measurements are useful for pre-treatment and post-treatment comparisons.

The shapable housing may be configured to function as a peripheral to conventional clinician trans-cutaneous nerve stimulation (TENS) equipment or TENS compatible equipment with TENS compatible power and TENS compatible controls, which may be programmed to implement some or all of the aforementioned functionalities, such as a system that is physically and programmably configured for use over the treatment area comprising joint articulations of the spinal column.

The “user” of the system may be an individual or a qualified health care professional. Programming of therapeutic modalities is preferably but optionally done by a qualified health care professional, such as a physician, acupuncturist, or physical therapist. Thus, the system is able to accommodate differences of professional opinion where professionals may choose differently as to shat type of modality may best serve a patient. The aforementioned telecommunications linkage may be used to access the Internet for purposes of visiting a health care website that contains a variety of medically approved program instructions for use in treating various conditions. Various modalities that may be implemented according to physician recommended protocols include bone problems, skin lesions, abscesses, ulcerations, tumors, breasts with fibrous density, scars from aspiration, biopsy scars, mastectomy scars, lactation promotion, nerve severance, nerve impingement, nerve inflammation, nerve disease, vascular occlusion, vascular compression, vascular stasis, lymphatic occlusion, lymphatic compression, lymphatic stasis, muscle injuries, tendon injuries, ligament injuries, soft tissue injuries, sports-induced fatigue, and carpel tunnel syndrome. Where appropriate, the modalites may be adjusted to address actual conditions, or for use as prophylactic treatments.

The shapeable housing may be provided with sensor devices that are configured to provide measurement signals indicating the efficacy of treatment, such as EMG or thermal sensors. Program instructions for the control mechanism may contain a biofeedback loop that is configured to interpret signals from the sensor devices and adjust a therapeutic protocol based upon interpretation of the sensed signals.

### BRIEF DESCRIPTION OF THE DRAWINGS

FIG. 1 depicts a therapeutic device system formed of a plurality of layers that include energy sources and a programmable control mechanism for implementing therapeutic modalities;

FIG. 2 is a schematic illustration of an expanded system incorporating a plurality of therapeutic devices like that shown in FIG. 1, all under the control of a single CPU;

FIG. 3 shows a therapeutic device system in a rigid casting construction deployed over a patient's knee;

FIG. 4 depicts a patient undergoing biofeedback-enhanced photonic therapy; and

FIG. 5 illustrates a biofeedback process for use in a programmable controller.

### DETAILED DESCRIPTION

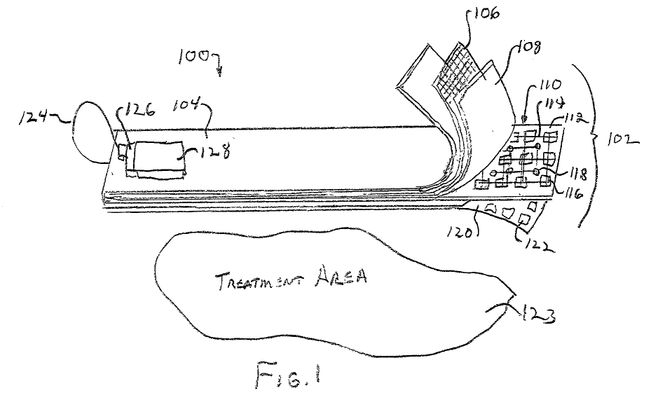

FIG. 1 is a perspective view of a patient or consumer therapeutic device system 100 that includes a plurality of laminated layers 102. In FIG. 1., the respective layers 102 are depicted as being partially peeled apart from one another, but this configuration is only for purposes of illustrating the interior portions of component layers 102 that collectively form a shapeable or selectively moldable housing. From top to bottom, the respective layers include an outer cover 104, which may be made from a variety of materials. Where the system 100 is desired to have a rigid shape, e.g., for casting purposes, the outer cover 104 may be made, for example, of a thermosetting resin, a plaster casting material, a composite resin casting material, or a curable polymer resin. The outer layer 104 is optionally cast to mirror a treatment area, such as a breast, knee, torso, neck, ear, or foot. These rigid materials are initially flexible for application purposes, but harden and convert to rigid form by the application of cooling temperatures, light, or chemical activity.

Where the system 100 is for temporary use that can always be flexibly deformed by manual manipulation, the outer cover 104 may be made, for example, of an elastic bandage material, latex, silicone, cloth, or any other material flexible material. A casting agent for covering wounds may include, for example, a flexible latex for conformable use, as an alternative to one of the more rigid materials identified above.

The shape of system 100 may be defined by a deformable memory-shape retention layer 106 that is made, for example out of a ductile metal, such as copper or a sheet of lead or aluminum foil, or a polymer putty. The purpose of shape retention layer 106 is to deform under manual manipulation for close fitting over a treatment area. Where the outer cover 104 is a material that is initially flexible and converts to a rigid form, such as a resin composite casting material, the shape retention layer 106 may be omitted, but use of the shape retention layer 106 is beneficial until such time as the resin composite cures into a rigid or hardened form. A flexible insulating layer 108 prevents electrical contact between the shape-retention layer 106 and the underlying energy source layer 110.

The energy source layer 110 may be, for example, a latex or silicone material 112 that is processed using printed circuit board techniques to produce a power grid 114 that permits selective individual operation of individual energy sources, such as a transcutaneous electrostimulation pad 116 and a LED or laser diode 118. The scale, number, and disposition of the energy sources 116, 118, within the energy source layer 110 may be any disposition having therapeutic utility. Generally, a smaller scale of energy sources and a finer power grid are preferred because the finer scale incorporates a greater number of such energy sources and generally increase the overall flexibility of system 100. For example, a particularly preferred form of diode for use as LED 118 are the microdiodes available from Panasonic, e.g., as part number LN 1261CAL emitting at 660 nm. Use of these microdiodes permits, for example, overall thicknesses down to one-sixteenth of an inch. Other diodes or microdiodes may be used. For example, those emitting in the blue ultraviolet range may be used to treat infections, or diodes emitting at 880 nm have also been proven to have therapeutic utility. Any emission wavelength having a perceived therapeutic benefit may be employed. Generally, the LEDs 118 may be selected to have a plurality of emission wavelengths for any therapeutic purpose, and these diodes may optionally include LEDs or laser diodes that emit light in any portion of the visible or nonvisible spectrum. The LEDs 118 are mounted in conventional receptacles (not shown) that facilitate their operation.

The transcutaneous electrostimulator pads 116 are exposed at the bottom of material 112. A contact layer 120 is made of transparent silicone or other flexible transparent material, and covers the bottom of the energy source layer 110. The contact layer 120 may contain a plurality of apertures, such as aperture 122 in alignment with the transcutaneous electrostimulator 116, to permit electrical contact between the transcutaneous electrostimulator 116 and a treatment area 123. Alternatively, the aperture 122 may be filled with an electrical contact in electrical communication with the transcutaneous electrostimulator 116.

A plug connector 124 includes a wiring array in contact with the power grid 112 and a portable power control mechanism for individual operation of the respective energy sources 116, 118 within energy source layer 110. The portable power control mechanism 126 may be housed, for example, in a pocket 128 built into the outer cover 104.

As shown in FIG. 1, the therapeutic device system 100 has a rectangular configuration that may, for example, be used to wrap a knee, neck or shin. The overall shape may vary to have any shape or construction that is compatible with any portion of anatomy or wound. The shape may be custom molded or generically sized for any application, such as a knee brace or torso cover. Additional devices for recording purposes (not shown) may be incorporated into the power grid 112, e.g., for sensing and reporting of electromyographic data representing reporting of the electrical skin conductivity of the treatment area, or for thermographic reporting and recording. This data is useful for comparison purposes that may indicate to medical personnel the efficacy of treatment or a need to alter treatment modalities. These modality alterations may also be programmed into program instructions within the control mechanism 126 for alteration based upon sensed data.

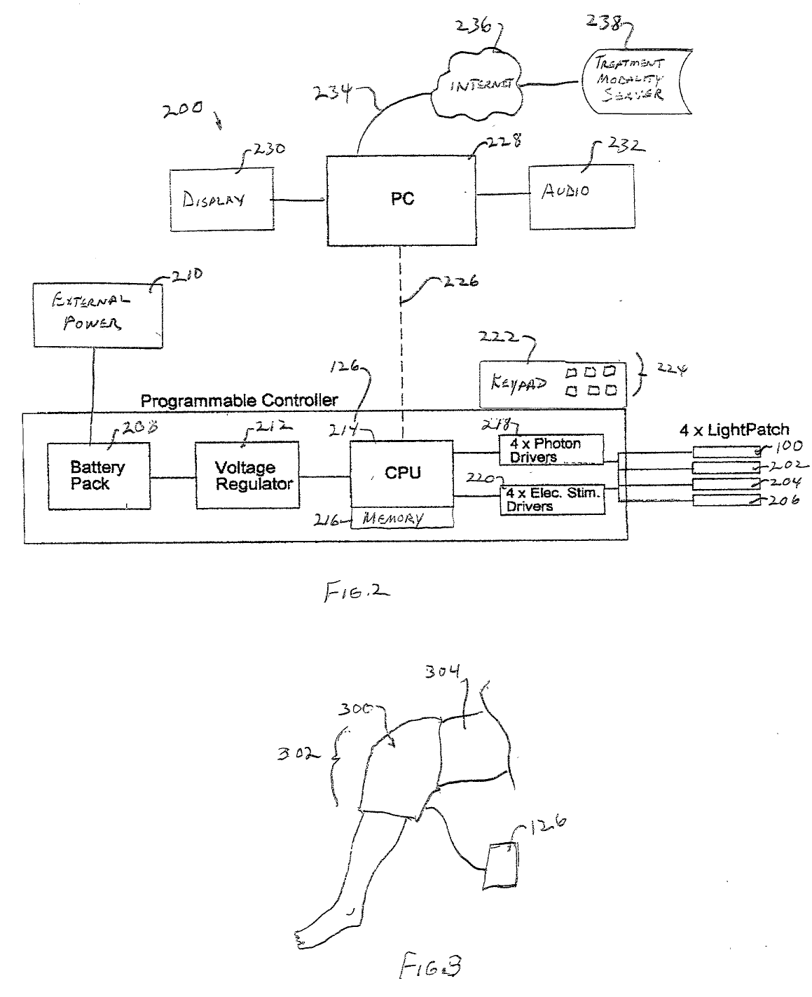

FIG. 2 schematically depicts an enhanced system 200 that contains the therapeutic device system 100 in combination with a plurality of identical therapeutic device systems 202, 204, and 206. FIG. 2 provides additional detail with respect to the power control mechanism 126. A battery pack 208 provides power to the system 200, and this power may be supplemented by connection to an external power source 210. A voltage regulator 212 functions to evenly distribute power to the respective energy sources 116,118 (shown in FIG. 1) according to the type of energy source. Selective application of power from the voltage regulator 212 is governed by a CPU 214 that operates based upon program instructions which are accessed from program memory 216. Photon drivers 218 and electrical stimulation drivers 220 apply power from the voltage regulator 212 based upon control instructions from CPU 214 and, consequently, operate each of the therapeutic device systems 100, 202, 204, and 206, all according to individually selectable therapeutic modalities. These drivers 218, 220 permit operation of the energy sources on an individual basis, or in banks of sources according to source type, e.g., in four banks of diodes emitting at different wavelengths or different intensities. A keypad 222 with user-selectable buttons 224 permits a user to define or select emission wavelengths, laser or LED light, Joule intensity emission standards, waveform functions, therapeutic modalities, electrostimulation current, electrostimulation voltage, TENS compatibility protocols, duration of elapsed treatment, multiple combined treatment modalities, and any other condition affecting treatment.

The control mechanism 126 is optionally connected by communications link 226, which may be a radio linkage or direct line, to a personal computer (PC) 228. The PC 228 is programmed with an interface to control mechanism that permits the PC 228 to visually display 230 the status of the respective treatment modalities being implemented by the control mechanism 126. The PC 228 also records the treatment sessions for medical record keeping and billing purposes. An audio speaker 232 announces, at periodic intervals, the progress of respective therapies in progress for review by patients and medical personnel alike, and announces an audible alarm if system 200 diagnoses a therapy or system problem, such as a depleted battery pack 208. The PC 226 may be located remotely from the control mechanism 126, and is able to provide CPU 214 with control instructions and to receive data from CPU 214 for operation of system 200 even at large distances, such as may be implemented by a telephone network. PC 228 is able to download program instructions including selected therapeutic modalities to CPU 214 and memory 216.

A telecommunications linkage 234 optionally connects PC 228 to the Internet 236, which may be used to access a host server 238 that functions as a central repository for distribution of program instructions and data that are related to therapeutic modalities. The host server 238 may also provide information concerning treatment options with success/failure studies or statistics regarding the various options. Thus, a patient may be able to review these statistics and reports and decide upon a particular modality from among a variety of modalities that may be used to address a given condition.

FIG. 3 illustrates a therapeutic system 300 that is deployed as a rigid cast over a treatment area 302 comprising a knee on leg 304. Control mechanism 126 is configured by program instructions to implement a treatment modality addressing natural healing process for a surgical wound on the treatment area 302 that is complicated by an infection.

In operation, the therapeutic benefit for a living body is obtained through selective or collective configuration of, and separate or simultaneous applications of, photon emissions and/or transcutaneous electrical stimulation using therapeutic systems 100, 200, or 400. The therapeutic benefit is selectively refined and enhanced through specific alterations of the photon emissions and/or specific alterations of the transcutaneous electrical stimulation, and/or through alterations of the exposure time and/or sequencing of the therapeutic events. Alterations of photon emissions may include the utilization of singular or multiple wave lengths, the coherent or non-coherent nature of the photon emission, the amount of electrical current catalyzing the photon emission, the wave form(s) of the electrical current effecting photon emission, and the constant and/or interval frequency(s) of the photon emissions. Alterations of the transcutaneous electrical stimulation may include the amount of (micro- or macro-) electrical current effecting trans-cutaneous stimulation, the wave form(s) of the electrical current effecting the trans-cutaneous stimulation, and/or the constant and/or interval frequency(s) of trans-cutaneous electrical stimulation. The therapeutic benefit for a living body can further be enhanced through incorporation of mechanisms for reporting and, thereby, facilitating and optimizing equipment utilization and treatment outcome. Such mechanisms for enhancement of treatment outcome may include means for reporting total elapsed time of current treatment and the total cumulative treatment time for the day, week and month vis-à-vis recommended and/or prescribed therapeutic objectives.

Therapeutic benefit for a living body can further be enhanced through the incorporation of mechanisms that report the multiple therapeutic events which are occurring simultaneously or cumulatively. Such mechanisms invite the user's subjective awareness and intentional therapeutic involvement, thereby enhancing treatment outcome. Mechanisms for enhancing conscious subjective therapeutic involvement may include visual reflection of photon frequency activity, interval auditory reminder(s) of device activity status, electromyographic reporting of electrical skin conductivity, and/or thermographic reporting of skin temperature, for pretreatment, during treatment, and/or post-treatment comparisons.

Design variations may be configured as either clinician-administered therapeutic devices, patient-operated devices, technician-administered devices, or as consumer-operated devices. All devices may have programmable options for system upgrades. Clinician-administered therapeutic devices will allow the clinician to select and program operational variables. Clinicians may elect to program the operational variables for prescribed or recommended patient-operated devices, or patient-operated devices may have pre-set operational variables for clinicians not wanting to alter default settings. All design variations may either have pre-set operational variables and/or interface with a central programming unit, and/or have such device communicative interfaces as infrared beams, and/or interface with computers via software, for direct or remote selection and regulation of therapeutic variables, and/or for direct or remote recording of equipment utilization variables. Invention design variations include control units of varying complexity, with clinician- and technician-administered models having the greatest flexibility in selecting operational variables.

Control mechanism 126 may regulate a therapeutic device system 100 having specialized forms, such as generic pads or braces of preconfigured dimensionality, or customized therapeutic pads, braces or casts, and/or therapeutic beds for consumer, commercial, and clinical use. Customized pads may mirror templates of surgical scars, disease scars, or trauma-induced scars, skin lesions, abscesses, ulcerations, tumors, or cysts. Larger versions may mirror entire zones of the living body, as in customized pads that mirror breast(s), e.g., for prevention and/or treatment of fibrous density of breasts, or for treatment and reduction of scar tissue, and scar numbness or increased sensitivity, from aspiration, biopsy, or partial or complete breast mastectomy, or for promotion of lactation. Custom or generically sized seat cushions may be provided, e.g., for prevention and/or treatment of ulcerations in the wheelchair-limited or bedridden patients. Custom pads may be made for treating joints, or areas of limbs, or of torsos, that have been strained or sprained, or for enhancing physiological functions of organs, or organ systems, including the regeneration of central and/or peripheral nerve severance, impingement, inflammation, or disease, and including prevention or treatment of occlusion, compression, stagnation, engorgement, or stasis of the vascular and/or lymphatic systems.

Customized braces may be configured for prevention or treatment of repetitive motion trauma, such as carpel tunnel syndrome, or sports-induced fatigue, strains, or sprains, or customized casts may be configured for treatment of diseased, fractured or broken bones, or for treatment of bulging or herniated spinal discs, or for treatment of severe strains or sprains, as in whiplash injuries. Generic pads and braces may be designed to meet averaged dimensional needs of clinicians, technicians and consumers. Invention design variations, such as therapeutic beds, wheelchair support cushions, massage tables, and/or physical medicine rehabilitation equipment, will support systemic neurological, vascular, and lymphatic circulatory enhancement of the living body.

The system 200 utilizes photon-emitting diodes, with or without transcutaneous electrical stimulating contact(s), embedded in flexible-to-rigid housing, conforming to or interacting with an animal or human body, i.e., a living body. The system design enables the energy sources to function as an integrated whole, or for housing(s) of photon-emitting diodes, with or without transcutaneous electrical stimulating contacts, to function independently, as peripherals, which can be plugged into existing clinician trans-cutaneous nerve stimulation (TENS) equipment. The flexibility or rigidity of housing(s) involve material means with memory retention, enabling the invention to:

(a) mechanically conform to and, where there is joint articulation, flex with anatomical structures,

(b) mechanically restrict or eliminate ranges of motion of these anatomical structures, or

(c) mechanically allow for a part or the entire living body to be contained within the therapeutic device system.

The system may utilize template-customization of shapable housings to mirror surgical, disease, or trauma-induced scars, skin lesions, abscesses, ulcerations, tumors, cysts, or, in much larger versions, mirror zones of the living body, as in customized pads for prevention and/or treatment of fibrous density of breasts, or for treatment and reduction of scar tissue, scar numbness, and/or scar hypersensitivity secondary to aspiration, biopsy, or partial or complete breast mastectomy.

Power is by direct current, such as a battery, and/or by alternating current. The central processing unit 214 enables local or remote programming and recording of modality sequencing, exposure time, micro- or macro-electrical current, wave form(s), frequency modulation, light wave length, and joules of light exposure.

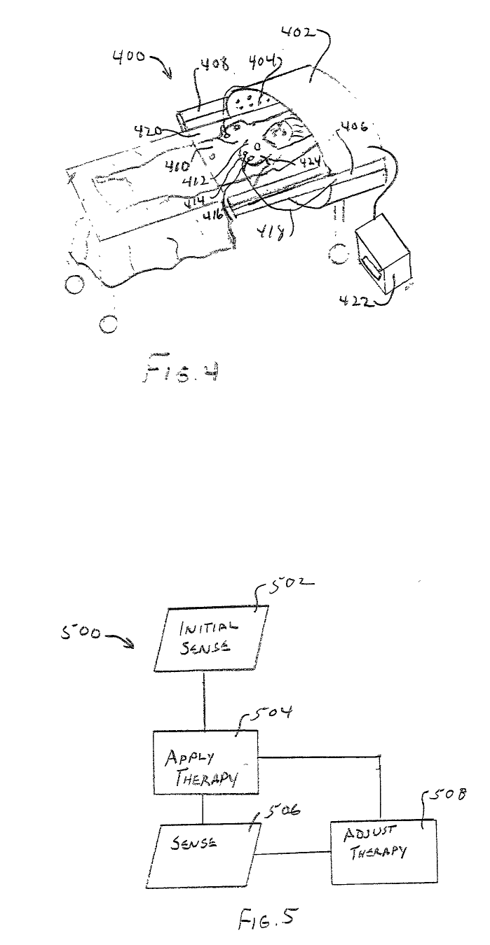

FIG. 4 depicts another embodiment, namely a bed device 400 having an ovaloid housing 402 that contains a plurality of LEDs or laser diodes 404. The ovaloid housing 402 is fore and aft shiftable on rails 406, 408 to selectively position the ovaloid housing 402 over a patient 410. As shown in FIG. 4, the patient 410 is ready to receive photonic treatment for a fibroid breast condition, e.g., in breast 412. An EMG sensor 412 and a thermal sensor 414 are coupled to breast 412 to sense temperature changes and naturally occurring electrical discharges. The sense measurements are indicators of circulation and/or muscle contracture. Lead 418 connects the EMG sensor 414 and the thermal sensor 416 with the ovaloid housing 402. Table 420 is transparent so that patient 410 may benefit from 360° exposure to impinging photons or light emanating from the ovaloid housing 402. A programmable controller 422 operates as described above for control mechanism 126, which may optionally administer electrotherapy through an acupuncture needle or transcutaneous electrostimulator pad 424. The therapeutic device 100 may be draped over a support frame (not shown in FIG. 4) to form the ovaloid housing 402.

The controller 422 is provided with program instructions implementing a biofeedback loop 500, as shown in FIG. 5. As a therapy session begins for patient 410, the ovaloid housing 402 is selectively positioned over patient 410 to establish a treatment area over and beneath breast 412. In step 502, initial sense measurements are obtained from the EMG sensor 414 and the thermal sensor 416 to establish a baseline. The programmable controller 406, in step 504, administers therapy according to any therapeutic protocol that is compatible with the ovaloid housing 402 and the condition of breast 412. In step 506, periodically or continuously during the therapy application step 504, sense measurements are again obtained from the EMG sensor 414 and the thermal sensor 416. In step 508, the programmable controller 422 interprets these sense measurements from step 506 and adjusts the therapeutic protocol, e.g., by altering the intensity or waveform of photons emanating from LED 404 and/or the waveform, voltage or current from the transcutaneous electrostimulator pad 424. The biofeedback loop 500 may be incorporated in program instructions for the CPU 214 shown in FIG. 2.

Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

### References

The following references pertain to the field of photonic therapy and electrostimulation therapy, and are hereby incorporated by reference.

| U.S. Pat. No. | Issue Date | 1^{st} Inventor |
|---|---|---|
| 6,156,028 | December 5, 2000 | Prescott. |
| 6,107,466 | August 22, 2000 | Hasan et al. |
| 6,099,554 | August 8, 2000 | Nordquist et al. |
| 6,096,066 | August 1, 2000 | Chen et al. |
| 6,074,411 | June 13, 2000 | Lai et al. |
| 6,063,108 | May 16, 2000 | Salansky et al. |
| 6,045,575 | April 4, 2000 | Rosen et al. |
| 5,957,569 | December 7, 1999 | Chen et al. |
| 5,957,960 | September 28, 1999 | Chen et al. |
| 5,944,748 | August 31, 1999 | Mager et al. |
| 5,913,883 | June 22, 1999 | Alexander et al. |
| 5,876,427 | March 2, 1999 | Chen et al. |
| 5,843,074 | December 1, 1998 | Cocilovo |
| 5,800,479 | September 1, 1998 | Thiberg |
| 5,779,483 | July 14, 1998 | Cho |
| 5,776,233 | June 16, 1998 | Thiberg |
| 5,616,140 | April 1, 1997 | Prescott |
| 5,549,660 | August 27, 1996 | Mendes et al. |
| 5,464,436 | November 7, 1995 | Smith |
| 5,385,503 | October 25, 1994 | Bertwell et al. |
| 5,304,207 | April 19, 1994 | Stromer |
| 5,300,097 | April 5, 1994 | Lerner et al. |
| 5,272,716 | December 21, 1993 | Soltz et al. |
| 5,259,380 | November 9, 1993 | Mendes et al. |
| 5,150,704 | September 29, 1992 | Tatebayashi et al. |
| 5,024,236 | June 18, 1991 | Shapiro |
| 4,930,504 | June 5, 1990 | Diamantopoulos et al. |
| 4,676,246 | June 30, 1987 | Korenaga |
| 4,535,784 | August 20, 1985 | Rohlicek et al. |

What is claimed is:

1. A therapeutic device system for therapeutic application of energy to a living body, comprising:
(a) energy sources selected from the group consisting of photon-emitting diodes, and photon emitting diodes in combination with trans-cutaneous electrical stimulators;
(b) a power grid adapted to provide electrical current for operation of the energy sources; and
(c) a shapable housing for the energy sources that permits connectivity between the energy sources and the power grid, the shapable housing being selectively moldable to retain a shape configuration when adapted conformably over a treatment area and placed on the living body.

2. The system as set forth in claim 1, wherein the shapable housing comprises a flexible material conforming to the treatment area.

3. The system as set forth in claim 2, wherein the flexible material comprises a metal sheet or mesh.

4. The system as set forth in claim 1, wherein shapable housing comprises a curable material that is flexible until cured and, after curing, comprises a substantially rigid material that provides support to the living body.

5. The system as set forth in claim 4, wherein the curable material comprises a comprises a thermosetting resin that is flexible when heated to a temperature above a design temperature and cures to rigidity when cooled to a temperature below the design temperature.

6. The system as set forth in claim 1, wherein the shapable housing comprises means for altering flexibility of the housing from a substantially rigid state to a substantially flexible state.

7. The system as set forth in claim 6, wherein the shape of the shapable housing is adapted to implement a therapeutic casting modality for immobilizing a portion of the living body.

8. The system as set forth in claim 1, wherein the shapable housing comprises a material with shape-memory retention, such that the material is capable of being reshaped to a neutral state after treatment on the living body is concluded.

9. The system as set forth in claim 8, wherein the material with shape-memory retention is selected fro the group consisting of manually deformable materials, heat setting materials, and chemical setting materials.

10. The system as set forth in claim 1, wherein the energy sources comprise both photon-emitting sources and trans-cutaneous electrical stimulators.

11. The system as set forth in claim 1, wherein the photon-emitting sources are embedded in the shapable housing formed of an insulating material to enhance heat concentration over the treatment area.

12. The system as set forth in claim 1, wherein the photon-emitting sources are embedded in the shapable housing formed of a heat conductive material to dissipate heat over the treatment area.

13. The system as set forth in claim 1, wherein the photon-emitting sources are covered by an optically transparent protective layer that provides a surface interfacing with the skin which can be cleaned and disinfected.

14. The system as set forth in claim 1, wherein the shapable housing is adaptable to a portion of the living body.

15. The system as set forth in claim 1, wherein the shapable housing comprises an adhesive layer to promote contact with the living body.

16. The system as set forth in claim 1, wherein the shapable housing is adapted on a customized basis to mirror individualized templates taken from the group consisting of surgical wounds; surgical scars; trauma-induced scars; skin lesions; abscesses; ulcerations; tumors; cysts; and physiological abnormalities related to soft-tissue, organ, lymph, neurological or vascular compromise of the living body.

17. The system as set forth in claim 1, wherein the shapable housing is adapted on a custom basis to mirror individualized templates taken of anatomical zones of the living body selected from the group consisting of breasts, joints, limbs, neck, and the torso.

18. The system as set forth in claim 1, wherein the shapable housing is generically-sized for treating conditions taken from the group consisting of surgical wounds; trauma-induced scars; skin lesions; abscesses; ulcerations; tumors; cysts; and physiological abnormalities related to soft-tissue, organ, lymph, neurological or vascular compromise of the living body.

19. The system as set forth in claim 1, wherein the shapable housing is generically-sized for adapting to anatomical features taken from the group consisting of breasts, joints, limbs, neck, and the torso.

20. The system as set forth in claim 1, wherein the shapable housing to serve as a device selected from the group consisting of a wheelchair cushion, an automotive seat cover, a mattress, a bedcover, and seat cover for a chair.

21. The system as set forth in claim 1, wherein the shapable housing is configured for entry into an enclosure of the living body.

22. The system as set forth in claim 21, wherein the shapable housing is adapted to provide three-dimensional exposure to the energy sources.

23. The system as set forth in claim 1, further comprising a connection to a power source capable of activating the energy sources.

24. The system as set forth in claim 23, wherein the power source is contained in the shapable housing.

25. The system as set forth in claim 24, wherein the power source comprises a battery.

26. The system as set forth in claim 25, wherein the battery comprises a flexible structural composition that is conformable to the treatment area.

27. The system as set forth in claim 23, further comprising a voltage regulator operable for uniform distribution of electrical current to the energy sources.

28. The system as set forth in claim 23, wherein the power grid is embedded in the shapable housing.

29. The system as set forth in claim 1, further comprising a control mechanism for regulation of output from the energy sources.

30. The system as set forth in claim 29, wherein the control mechanism comprises manual switches for triggering operation of program instructions processed by a central processing unit (CPU).

31. The system as set forth in claim 30, comprising programmable memory operably coupled with the CPU.

32. The system as set forth in claim 31, wherein the programmable memory contains program instructions for a variety of therapeutic modalites that may be selectively accessed via the manual switches according to protocols for treating a variety of conditions through use of the energy sources.

33. The system as set forth in claim 32, including a telecommunications linkage for selecting a therapeutic modality and retrieving a record of the therapeutic modalities that have been implemented on the living body

34. The system as set forth in claim 32, wherein the program instructions define combined treatment modalities that sequence different pattern activation of the energy sources within a single therapeutic application.

35. The system as set forth in claim 32, comprising a plurality of the shapable housings and wherein the program instructions permit the control mechanism to implement different treatment modalities for respective shapable housings.

36. The system as set forth in claim 32, wherein the program instructions selectively define total elapse exposure time of the energy sources.

37. The system as set forth in claim 32, wherein the program instructions permit a user to define a wave form of electrical current energizing the energy sources.

38. The system as set forth in claim 32, wherein the program instructions permit a user to define the frequency modulation (Hz) of the energy sources within the shapable housing.

39. The system as set forth in claim 32, wherein the energy sources comprise trans-cutaneous electro stimulators, and the program instructions permit a user to select whether the trans-cutaneous electro stimulators use micro-electrical current or macro-electrical current to energize the trans-cutaneous electro-stimulators.

40. The system as set forth in claim 32, wherein the program instructions permit a user to select the type of energy sources for activation within the shapable housing.

41. The system as set forth in claim 32, wherein the program instructions permit a user to select wavelengths for emission upon activation of a corresponding portion of the energy sources.

42. The system as set forth in claim 32, wherein the program instructions permit a user to selectively define the milliwatts of electrical current applied to the respective energy sources.

43. The system as set forth in claim 32, wherein the program instructions permit a user to select the joules of photon emission from the energy sources.

44. The system as set forth in claim 32, wherein the control mechanism comprises a visual display configured to provide visual confirmation of the selected program instructions.

45. The system as set forth in claim 32, wherein the control mechanism comprises means for generating interval auditory reminders of the system activity status according to the program instructions.

46. The system as set forth in claim 1, comprising means for electromyographic reporting of the electrical skin conductivity of the treatment area.

47. The system as set forth in claim 1, comprising means for thermographic reporting of skin temperature alterations over the treatment area for use in pre- and post-treatment comparisons.

48. The system as set forth in claim 1, wherein the shapeable housing is configured to function as a peripheral selected from the group consisting of clinician trans-cutaneous nerve stimulation (TENS) equipment and TENS compatible equipment having TENS compatible power and TENS-compatible control functions.

49. The system as set forth in claim 1, wherein the shapeable housing is physically and programmably configured for use over the treatment area comprising joint articulations of the spinal column.

50. The system as set forth in claim 1, wherein the shapeable housing is physically and programmably configured for use over a treatment area comprising a bone.

51. The system as set forth in claim 1, wherein the shapeable housing is physically and programmably configured for use over a treatment area selected from the group consisting of skin lesions, abscesses, ulcerations, and tumors.

52. The system as set forth in claim 1, wherein the shapeable housing is physically and programmably configured for use over a treatment area comprising breast tissue that bears a harm selected from the group consisting of fibrous breast density, scars from aspiration, biopsy scars, mastectomy scars, skin lesions, abscesses, ulcerations, and tumors.

53. The system as set forth in claim 1, wherein the shapeable housing is physically and programmably configured for use over a treatment area comprising breast tissue, and the system includes means for operating the system according to a protocol for promotion of lactation.

54. The system as set forth in claim 1, wherein the shapeable housing is physically and programmably configured for use over a treatment area comprising a harm selected from the group consisting of nerve severance, nerve impingement, nerve inflammation, and nerve disease.

55. The system as set forth in claim 1, wherein the shapeable housing is physically and programmably configured for use over a treatment area comprising a vascular system with a harm selected from the group consisting of occlusion, compression, and stasis.

56. The system as set forth in claim 1, wherein the shapeable housing is physically and programmably configured for use over a treatment area comprising a lymphatic system with a harm selected from the group consisting of occlusion, compression, and stasis.

57. The system as set forth in claim 1, wherein the shapeable housing is physically and programmably configured for use on injuries selected from the group consisting of injuries to muscle, tendon, ligaments, and soft-tissue.

58. The system as set forth in claim 1, wherein the shapeable housing is physically and programmably configured for use against repetitive motion trauma selected from the group consisting of carpel tunnel syndrome, sports-induced fatigue, strains, and sprains of the living body.

59. The system as set forth in claim 1, wherein the shapeable housing is physically and programmably configured for prophylactic use against conditions selected from the group consisting of repetitive stress disorders, sports-induced fatigue, strains, and sprains of the living body.

60. A method of providing therapy by the action of energy sources, the method comprising the steps of:
conforming to contours defined by a treatment area on a living body a shapable housing for energy sources and a power grid, the shapable housing being molded by this confirming step to self-retain a shape configuration through the use of memory shape-retention means; and
activating the energy sources selected from the group consisting of photon-emitting diodes, and photon emitting diodes in combination with trans-cutaneous electrical stimulators, according to a therapeutic protocol..

61. The method as set forth in claim 60, including a step of adjusting a therapeutic modality based upon biofeedback information that indicates the efficacy of treatment.

62. In a therapeutic device having a programmable controller and energy sources selected from the group consisting of LEDs. laser diodes, and electrostimulation devices, and combinations thereof, the improvement comprising:
sensor devices configured to provide measurement signals indicating the efficacy of treatment, and
a biofeedback loop configured to interpret signals from the sensor devices and adjust a therapeutic protocol based upon interpretation of the signals.
